(19) United States
(12) Patent Application Publication
(10) Pub. No.: US 2016/0008256 A1
(43) Pub. Date: Jan. 14, 2016

(54) A PHOTOPROTECTIVE PERSONAL CARE COMPOSITION

(71) Applicant: CONOPCO, INC., D/B/A UNILEVER, Englewood Cliffs, NJ (US)

(72) Inventors: Sudipta GHOSH DASTIDAR, Bangalore (IN); Bharath PALANISAMY, Bangalore (IN)

(73) Assignee: Conopco, Inc., d/b/a UNILEVER, Englewood Cliffs, NJ (US)

(21) Appl. No.: 14/771,616

(22) PCT Filed: Feb. 17, 2014
(86) PCT No.: PCT/EP2014/052999

§ 371 Date: Aug. 31, 2015

(30) Foreign Application Priority Data
Mar. 8, 2013 (EP) 13158333.8

(51) Int. Cl. A61K 8/81; A61K 8/37; A61Q 17/04; A61K 8/02; A61K 8/29

(52) U.S. Cl. CPC A61K 8/8147; A61K 8/0279; A61K 8/29; A61Q 17/04; A61K 8/37; A61K 2800/412; A61K 2800/592

(57) ABSTRACT

The invention relates to a photo-protective personal care composition that provides high sun protection factor (SPF). It has been found by way of the present invention that specific non-ionic surfactants interact synergistically with hollow microspheres in a cosmetic base comprising low amount of fatty acid to enhance the SPF of low organic sunscreen containing composition.

### FIELD OF THE INVENTION

The invention relates to a photo-protective personal care composition that provides high sun protection factor (SPF).

### BACKGROUND OF THE INVENTION

Solar radiation includes about 5% ultraviolet (UV) radiation, wavelength of which is between 200 nm and 400 nm. It is further classified into three regions: from 320 to 400 nm (UV-A), 290 to 320 nm (UV-B) and from 200 to 290 nm (UV-C). A large part of UV-C radiation is absorbed by the ozone layer. Scientific studies have indicated that exposure to UV-A and UV-B radiation for short period causes reddening of the skin and localized irritation, whereas continued and prolonged exposure can lead to sunburn, melanoma and formation of wrinkles. It is also reported that UV radiation causes significant damage to hair. Therefore, it is desirable to protect the skin and other keratinous substrates of the human body from the harmful effects of both, UV-A and UV-B radiation.

Various cosmetic preparations have been reported for preventing and/or protecting the skin from harmful effects of ultraviolet radiation. Many organic sunscreen agents capable of absorbing UV-A rays are reported in the field of cosmetics. Many UV-B sunscreens are also known and approved for safe use in personal care compositions for protection from UV-B radiation. Cosmetic manufacturers prefer to include both UV-A and UV-B sunscreens in photoprotective compositions so as to provide protection over the entire range of UV radiation. Sun protection Factor (SPF) and UV-A protection factor (UVAPF) are commonly measured attributes of photoprotective compositions which indicate the protection that the skin gets from exposure to both UV-B and UV-A radiation.

Cosmetic manufacturers try to provide consumers with products having higher and higher SPF/UVAPF. One of the ways of achieving this is to incorporate high levels of UV-A and UV-B sunscreens. One disadvantage of this approach is the high cost associated with incorporation of high levels of sunscreens which are expensive.

Further, there are safety and regulatory limitations on the upper limit of incorporation of these sunscreens. Sensory properties are also reported to get affected on incorporation of high levels of sunscreens. Hence, there is a problem of achieving high SPF/UVAPF while keeping the total amount of sunscreens in the compositions relatively low.

The present inventors have been working towards achieving high SPF in low organic sunscreen containing compositions. After extensive experimentation, they have found that a combination of two seemingly unrelated materials, one a specific type of non-ionic surfactant and the other a hollow microsphere when incorporated in a low fatty acid containing cosmetic composition, are able to synergistically interact to provide high SPF in low organic sunscreen containing composition.

US2009162443 (Schering Plough) discloses moisturizing compositions comprising microspheres for the purpose of preventing or reducing moisture loss from the skin.

US2005031655 (Schering Plough) discloses an emulsion composition for skin application which comprises an aqueous component, one or more water-insoluble organic components totaling at least about 15 percent by weight of the composition, and about 2 to about 10 percent by weight of a solid component consisting of porous silica microspheres having an average particle size between about 5 urn and about 20 μm.

US2012015016 (L'Oreal) discloses a UV protecting composition containing: (a) at least one organic UV sunscreen active; (b) at least one semi-crystalline polymer which is solid at ambient temperature and has a melting point of less than about 80° C.; (c) hollow latex particles; and (d) at least one additional ingredient chosen from: i) a UV light absorbing compound having an SPF of less than 2, and ii) an SPF booster capable of reflecting UV light, different from hollow latex particles, wherein the hollow latex particles are employed in an amount equal to, or greater than, the amount of the at least one organic UV sunscreen active present in the composition.

WO12084442 (Unilever) relates to a high SPF sunscreen composition comprising dibenzoylmethane or its derivative in combination with an oil soluble UV-B sunscreen when incorporated in a sunscreen composition along with a non-ionic surfactant of a select class meeting certain HLB requirements, provide the enhanced SPF benefits when applied on the substrate of interest.

There are thus, the above listed prior publications in the field of skin cosmetic compositions some of which are directed to UV protection. It is to be noted that none of them have disclosed or taught that specific non-ionic surfactants interact synergistically with hollow microspheres in a cosmetic base comprising low amount of fatty acid to enhance the SPF of low organic sunscreen containing composition.

It is thus an object of the present invention to provide for a low organic sunscreen containing composition that exhibits high SPF.

It is another object of the present invention to provide this high SPF using ingredients that are commonly individually used but have not been known to be used together to synergistically interact to give this benefit and thereby this is achieved at low cost.

### SUMMARY OF THE INVENTION

The first aspect of the present invention relates to a photoprotective personal care composition comprising
(i) 0.1 to 15% organic sunscreen;
(ii) 0.1 to 5% non-ionic surfactant having an HLB value of at least 13;
(iii) 0.1 to 6% hollow microspheres; and
(iv) a cosmetically acceptable base comprising 1 to 8% fatty acid by weight of the composition.

Another aspect of the present invention relates to a method of providing enhanced SPF to skin comprising the step of applying a composition of the present invention on to the desired surface of skin.

Yet another aspect of the present invention relates to use of a composition as of the present invention for obtaining an SPF of at least 20.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages of the composition unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

By “A Photoprotective Personal Care Composition” as used herein, is meant to include a composition for topical application to sun-exposed areas of the skin and/or hair of mammals, especially humans. Such a composition may be generally classified as leave-on or rinse off, and includes any product applied to a human body for also improving appearance, cleansing, odor control or general aesthetics. It is preferably a leave-on product. The composition of the present invention can be in the form of a liquid, lotion, cream, gel, or toner, and may be applied with an implement or via a face mask, pad or patch. Preferably the composition is in the form of a lotion or gel. A particularly preferred example of such a composition is a leave-on skin lotions. “Skin” as used herein is meant to include skin on the face and body (e.g. neck, chest, back, arms, underarms, hands, legs, buttocks and scalp) and especially to the sun exposed parts thereof. The composition of the invention is also of relevance to applications on any other keratinous substrates of the human body other than skin e.g. hair where products may be formulated with specific aim of providing photoprotection.

By way of the present invention it is possible to prepare high SPF personal care compositions. By high SPF is meant a composition that has an SPF of at least 20, preferably at least 25, more preferably at least 35. This high SPF is achieved using low amount of total organic sunscreens in the range of 0.1 to 15%, preferably from 2 to 10%, more preferably 4 to 10% by weight of the composition.

The first aspect of the present invention relates to a photoprotective personal care composition comprising 0.1 to 15% organic sunscreen; 0.1 to 5% non-ionic surfactant having an HLB value of at least 13; 0.1 to 6% hollow microspheres; and a cosmetically acceptable base comprising 1 to 8% fatty acid by weight of the composition.

The composition is preferably a non-solid sunscreen composition. By a non-solid sunscreen composition is meant a composition that has a critical shear stress (apparent yield stress) of less than 100 Pa, preferably less than 20 Pa at 25° C. The apparent yield stress is preferably at least 5 Pa at 25° C.

It is preferred that the non-solid composition has a viscosity at critical shear stress of less 1000 Pa·s at 25° C. Critical shear stress (apparent yield stress) is as defined by H A Barnes (Handbook of Elementary Rheology, University of Wales Aberystwyth, 71-73 (2000)). A preferred non-soild composition is a lotion. Lotion prepared using the present invention preferably has a viscosity of 1500 to 6000 cP as measured by a Brookefield Viscosity meter using LV #4, 30 rpm, 30° C.

Definition of lotion has been given by Brummer (Rheology Essentials of Cosmetic and Food Emulsions, Springer-Verlag Berline Heidelberg, 81-83 (2006)). Therein lotions are those compositions which flow out from the container at 25° C. when turned upside down.

The invention provides for a high SPF sunscreen composition comprising 0.1 to 15% organic sunscreen. These organic sunscreens are preferably chosen from the following seven major groups: (1) benzophenones, (2) anthranilates, (3) dibenzoylmethanes (4) salicylates, (5) cinnamates, (6) camphores and (7) p-amino benzoic acid (PABA) or their derivatives or mixtures. The organic sunscreens may be of the UV-A or of the UV-B sunscreen types. Preferred UV-A sunscreen is a dibenzoylmethane, triazine, triazone, or benzophenone derivative. A more preferred UV-A sunscreen belongs to the dibenzoylmethane group. When present, this is included in 0.1 to 5% dibenzoylmethane or its derivative. Preferred dibenzoylmethane derivatives are selected from 4-tert-butyl-4′-methoxydibenzoylmethane, 2-methyldibenzoylmethane, 4-methyl-dibenzoyl-ethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane. The most preferred dibenzoylmethane derivative is 4-tert.-butyl-4′-methoxydibenzoylmethane. Dibenzoylmethane or its derivative is preferably present in 0.2 to 5%, more preferably 0.4 to 3% by weight of the composition.

The composition of the invention may comprise a UV-B sunscreen. UV-B organic sunscreen is preferably selected from the class of cinnamic acid, salicylic acid, diphenyl acrylic acid or derivatives thereof. A few of the preferred oil soluble UV-B sunscreens which are commercially available and useful for inclusion in the composition of the invention are Octisalate™, Homosalate™, NeoHelipan™′ Octocrylene™, Oxybenzone™ or Parsol MCX™. When present, UVB sunscreen is included in 0.1 to 7%, preferably from 0.5 to 6%, more preferably 1 to 5% by weight of the composition.

An important ingredient that contributes to enhancement of SPF of the sunscreen composition of the invention is a selective class of non-ionic surfactant which have an HLB value of at least 13. The non-ionic surfactant is preferably selected from the class of fatty alcohol ethoxylates, alkyl phenol ethoxylates and polyoxyethylene sorbitan alkyl esters which satisfy the above HLB criterion.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:

A value <10: Lipid soluble (water insoluble)
A value >10: Water soluble
A value from 4 to 8 indicates an anti-foaming atent
A value from 7 to 11 indicates a W/O (water in oil) emulsifier
A value from 12 to 16 indicates oil in water emulsion
A value from 11 to 14 indicates a wetting agent
A value from 12 to 15 is typical of detergents
A value of 16 to 20 indicates a solubiliser or hydrotrope

Non-ionic surfactant of the fatty alcohol ethoxylates which may be used in the present invention are sold under the brand names given below: Brij 35 (HLB of 16.9), Brij 700 (HLB of 18.8 and Brij 99 (HLB of 15.3).

Ethoxylates of fatty alcohol (usually available under the Brij class) have the general structure:

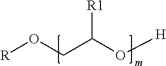

where R is a group consisting of carbon chain of 2 to 24; and R1 is alkyl or H; and m is a number from 1 to 100.

Ethoxylates of the fatty acid (usually available under the Myrj class) have the structure:

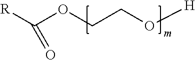

where R is a group consisting of carbon chain of 2 to 24; and m is a number from 1 to 100.

Non-ionic surfactant of the alkyl phenol ethoxylates which may be used in the present invention are sold under the brand names given below:

Triton X100 (HLB of 13.4), IGEPAL CA 720 (HLB of 14), Triton X 102 (HLB of 14.4), Triton X 165 (HLB of 15.5), Triton X 305 (HLB of 17.3), Triton X 405 (HLB of 17.6) and Triton X 705 (HLB of 18.4).

A typical structure of alkyl phenol ethoxylate is given below:

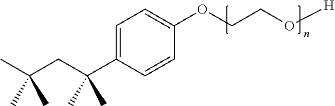

Where n is a number from 5 to 100.

Non-ionic surfactant of the polyoxyethylene sorbitan alkyl esters which may be used in the present invention are sold under the brand names given below:

Tween 60 (HLB of 14.9), Tween 21 (HLB of 13.1), Tween 80 (HLB of 15.0), Tween 40 (HLB of 15.6) and Tween 20 (HLB of 16.9).

The above class of non-ionic polymer viz. polyoxyethylene sorbitan alkyl esters have the general structure as given below:

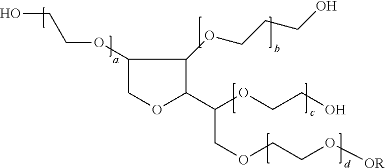

where R is long carbon chain.

The non-ionic surfactant is present in 0.1 to 5%, preferably 1 to 4% by weight of the composition.

The composition of the invention includes a hollow microsphere. The microspheres for use in the present invention are of the core shell type. The core is hollow, i.e it merely comprises air. By the term hollow is meant that the core of the microsphere is substantially free of any solid or liquid material. Preferably the core comprises more than 90 volume percent air, more preferably more than 95 volume percent air. Hollow microspheres are different from porous particles which do not have a core and shell configuration. By mean diameter is meant the number average mean diameter of the particles. In this specification, the particle size distribution of commercial particles were determined using Malvern particle size analyzer. The diameter of the hollow microspheres of the invention were determined using dynamic light scattering instrument (purchased from Brookhaven) which was coupled with a Lexel 95 laser (wavelength of 488 nm). The shell preferably has a thickness of 20 to 150 nm, more preferably 20 to 60 nm, further more preferably 20 to 30 nm. The hollow microspheres are preferably polymeric or inorganic in nature, more preferably polymeric. When it is inorganic, it may be a metal oxide preferably titanium dioxide, zinc oxide, tin oxide or cerium oxide more preferably titanium dioxide or zinc oxide. When it is polymeric, the microspheres are preferably made of polystyrene, polyacrylate or polystyrene co-polyacrylate. It is preferably polystyrene or a co-polymer thereof. The hollow microsphere preferably has an outer diameter in the range of 100 to 600 nm. The hollow polymeric microspheres preferably have an inner diameter in the range of 50 to 350 nm and an outer diameter in the range of 100 to 400. The hollow microspheres are preferably in the range of 1 to 5% by weight of the composition.

Without wishing to be bound by theory the inventors believe that the hollow microspheres with air inside provides the necessary mismatch of the refractive index between the core and the shell thereby increasing the path length of light which increases the possibility of light interacting with more of the sunscreens. In order to enable the above phenomenon effectively it is necessary to effectively disperse the microspheres which is enabled by the selective non-ionic surfactant which also additionally, optimally solubilises the sunscreen in the fatty acid matrix. All of the above factors are optimally enabled by way of the selective inclusion of the ingredients claimed in the present invention thereby providing the enhanced sun-screening efficacy.

The composition of the invention comprises a cosmetically acceptable base. The cosmetically acceptable bases are such as to have a product in preferably a non-solid form e.g. a lotion or emulsion format. The non-solid form may however be thickened through use of thickeners to form a gel. The composition preferably comprises 3 to 8% fatty acid. C12 to C20 fatty acids are especially preferred, further more preferred being C14 to C18 fatty acids. The fatty acid is preferably substantially a mixture of stearic acid and palmitic acid. A mixture of stearic acid and palmitic acid in weight ratio of 55:45 is known as hysteric acid. The cosmetically acceptable base is usually from 10 to 99.9%, preferably from 50 to 99% by weight of the composition. The cosmetically acceptable base preferably includes water. Water is preferably included in 35 to 90%, more preferably 50 to 85%, further more preferably 50 to 80% by weight of the composition.

Other useful sun-protective agents e.g. inorganic sunscreen may be preferably used in the present invention. These include, for example, zinc oxide iron oxide, silica, such as fumed silica, or titanium dioxide. The total amount of inorganic sunscreen that is preferably incorporated in the composition according to the invention is from 1 to 8%, preferably 3 to 8% by weight of the composition. It has been observed that inclusion of inorganic sunscreen provides further synergistic benefit in SPF in addition to the essential ingredients of the present invention.

The composition of the invention may additionally comprise a skin lightening agent. The skin lightening agent is preferably chosen from a vitamin B3 compound or its derivative e.g. niacin, nicotinic acid, niacinamide or other well known skin lightening agents e.g. aloe extract, ammonium lactate, azelaic acid, kojic acid, citrate esters, ellagic acid, glycolic acid, green tea extract, hydroquinone, lemon extract, linoleic acid, magnesium ascorbyl phosphate, vitamins like vitamin B6, vitamin B12, vitamin C, vitamin A, a dicarboxylic acid, resorcinol derivatives, hydroxycarboxylic acid like lactic acid and their salts e.g. sodium lactate, and mixtures thereof. Vitamin B3 compound or its derivative e.g. niacin, nicotinic acid, niacinamide are the more preferred skin lightening agent as per the invention, most preferred being niacinamide. Niacinamide, when used, is preferably present in an amount in the range of 0.1 to 10%, more preferably 0.2 to 5% by weight of the composition.

The composition according to the invention may also comprise other diluents. The diluents act as a dispersant or carrier for other materials present in the composition, so as to facilitate their distribution when the composition is applied to the skin. Diluents other than water can include liquid or solid emollients, solvents, humectants, thickeners and powders.

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry, which are suitable for use in the compositions of the present invention. Examples include: antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents, and skin healing agents.

Another aspect of the present invention relates to a composition of the invention for providing enhanced SPF to the skin by a method comprising the step of applying said composition onto desired surface of said skin.

Another aspect of the present invention relates to a method of providing enhanced SPF to skin comprising the step of applying a composition as claimed in any one of the preceding claims on to the desired surface of skin.

Yet another aspect of the invention relates to use of a composition of the invention for obtaining an SPF of at least 20, preferably at least 25, more preferably at least 35. The use is preferably for non-therapeutic benefits.

The invention is now further described by way of the following non-limiting examples.

### EXAMPLES

### Examples 1-3

### SPF of Compositions of the Invention as Compared to Outside the Invention

Compositions as shown in Table-1 were prepared. The in-vitro SPF of the compositions was measured using the following procedure:

Plates made of PMMA (7*7 cm ex. Shoenberg) was used as substrate to measure the SPF of the formulations. 2 mg/cm² of sample was dispensed uniformly on it as small drops using a syringe. By using Parafilm as finger coat, the sample was spread onto the transpore tape uniformly to get a uniform film. The film was allowed to dry for thirty minutes under ambient condition. After 30 minutes, the sample plate was exposed to UV lamp and transmittance spectrum was collected using SPF-290S spectrophotometer (ex. Optometrics Corporation). From transmittance spectrum obtained, the instrument calculates the in-vitro SPF values. For a single film, transmittance spectrum was collected at eight different places to get a statistical significance. The same was repeated for two more plate and thus the data reported is an average of 24 measurements. Glycerine applied PMMA was used as a reference substrate.

The SPF values measured are also summarised in Table-1 below.

TABLE 1

| | Example | | |
|---|---|---|---|
| | 1 | 2 | 3 |
| Ingredients | Wt % | Wt % | Wt % |
| Glycerine | 2.5 | 2.5 | 2.5 |
| Potassium | 0.2 | 0.2 | 0.2 |
| Hydroxide | | | |
| Cetyl alcohol | 0.37 | 0.37 | 0.37 |
| Isopropyl Myristate | 3 | 3 | 3 |
| Hystric acid | 7.0 | 7.0 | 7.0 |
| GMS NSE | 2 | 2 | 2 |
| (Glycerine mono | | | |
| stearate) | | | |
| Isopropyl myristate | 3 | 3 | 3 |
| DC 200,200 | 1 | 1 | 1 |
| (Silicone oil) | | | |
| Parsol MCX | 5 | 5 | 5 |
| Parsol 1789 | 3 | 3 | 3 |
| Brij 35 | 2.0 | 2.0 | 2.0 |
| Sunsphere** | 0.0 | 1.0 | 2.0 |
| Water | To 100 | To 100 | To 100 |
| SPF | 41.2 | 53.6 | 84.0 |

**Sunsphere is a hollow microsphere made of styrene/acrylates copolymer material with particle size in the range of 100 to 500 nm sourced from Dow Chemicals.

The data in Table-1 above indicates that compositions as per the invention (Examples 2 and 3) provide for vastly improved SPF as compared to that outside the invention.

### Example 4, 5

### Effect of Inclusion of Fatty Acid

Compositions as shown in Table-3 were prepared and the performance of the composition in its ability to filter light was measured using Absorbance technique. The Absorbance was measured using the following procedure:

Plates made of PMMA (7*7 cm ex. Shoenberg) was used as substrate to measure the SPF of the formulations. 2 mg/cm² of sample was dispensed uniformly on it as small drops using a syringe. By using Parafilm as finger coat, the sample was spread onto the transpore tape uniformly to get a uniform film. The film was allowed to dry for thirty minutes under ambient condition. After 30 minutes, the sample plate was exposed to UV lamp and the absorption spectrum was collected using SPF-290S spectrophotometer (ex. Optometrics Corporation).

The absorbance of the compositions at various wavelengths is also summarised in Table-2.

TABLE 2

Example

| | 4 | 5 |
|---|---|---|
| Ingredients | Wt % | Wt % |
| Glycerine | 2.5 | 2.5 |
| Hystric acid | 6.0 | 0.0 |
| Potasium Hydroxide | 0.2 | 0.2 |
| Cetyl alcohol | 0.37 | 0.37 |
| Isopropyl myristate | 1 | 1 |
| Glyceryl monostearate | 1.5 | 1.5 |
| Cetostearyl Ethylhexanoate (and) | 2 | 2 |
| Isopropyl Myristate (Crodamol | | |
| CAP) | | |
| Isohexadeccane (Arlamol HD) | 3 | 3 |
| Aluminum Starch Octenylsuccinate | 1 | 1 |
| (Dry flo PC) | | |
| Triethanol amine | 2.2 | 2.2 |
| Parsol MCX | 7.5 | 7.5 |
| Parsol 1789 | 3 | 3 |
| Tween 20 | 3.0 | 3.0 |
| Sunsphere | 5.0 | 5.0 |
| Ensulizole | 4 | 4 |
| Water | To 100 | To 100 |
| Absorbance, 300 nm | 3.93 | 3.15 |
| Absorbance, 320 nm | 3.86 | 2.67 |
| Absorbance, 340 nm | 2.19 | 1.62 |
| Absorbance, 360 nm | 2.17 | 1.61 |
| Absorbance, 380 nm | 1.83 | 1.40 |

The data in Table-2 above indicates that there is significant benefit in terms of increased absorbance in including fatty acid in the composition of the invention (Example 4) as compared to a composition without fatty acid (Example 5).

### Example 6

### Composition Including Inorganic Sunscreen

Composition as shown in Table 3 was prepared which contained high amount of inorganic sunscreen Titanium dioxide. The SPF was measured using the same procedure as described earlier for Example-1 and the data is summarised in Table-3 below.

TABLE 3

| | Example |
|---|---|
| | 4 |
| Ingredients | Wt % |
| Glycerine | 1.0 |
| Potassium hydroxide | 0.2 |
| Hystric acid | 6.0 |
| Sunsphere | 5.0 |
| Titanium dioxide (MT100Z) | 6.0 |
| Isopropyl myristate | 1.0 |
| Glyceryl monostearate | 1.5 |
| Ensulizole^{#} | 1.0 |
| Parsol MCX^{##} | 5.0 |
| Parsol 1789^{###} | 2.5 |
| Tween 20 | 4.0 |
| Sunsphere | 5.0 |
| Water | To 100 |
| SPF | 61 |

^{#}Ensulizole: Phenylbenzimidazole sulfonic acid ex. DSM
^{##}Parsol MCX: 2-ethylhexyl (E)-3-(4-methoxyphenyl)prop-2-enoate ex. Galaxy Surfactants
^{###}Parsol1789: 1-(4-tert-butylphenyl)-3-(4-methoxyphenyl)propane-1,3-dione ex. DSM

The data in Table-3 above indicates that a composition of the invention additionally comprising an inorganic sunscreen provides for high sun screening efficacy.

1. A photoprotective personal care composition comprising
(i) 0.1 to 15% organic sunscreen;
(ii) 0.1 to 5% non-ionic surfactant having an HLB value of at least 13;
(iii) 0.1 to 6% hollow microspheres; and
(iv) a cosmetically acceptable base comprising 1 to 8% fatty acid by weight of the composition.

2. A composition as claimed in claim 1 comprising 2 to 10% organic sunscreen.

3. A composition as claimed in any one of the preceding claims comprising 1 to 5% hollow microspheres.

4. A composition as claimed in any one of the preceding claims wherein said hollow microspheres are polymeric or inorganic.

5. A composition as claimed in claim 4 wherein said hollow microspheres are polymeric hollow microspheres.

6. A composition as claimed in claim 5 wherein said hollow polymeric microsphere comprises polystyrene or a co-polymer thereof.

7. A composition as claimed in any one of the preceding claims additionally comprising 1 to 8% inorganic sunscreen.

8. A composition as claimed in claim 7 wherein said inorganic sunscreen is present in 3 to 8% by weight of the composition.

9. A composition as claimed in claim 7 or 8 wherein said inorganic sunscreen is selected from titanium dioxide or zinc oxide.

10. A composition as claimed in any one of the preceding claims wherein the non-ionic surfactant is selected from the group consisting of fatty alcohol ethoxylates, alkyl phenol ethoxylates and polyoxyethylene sorbitan alkyl esters.

11. A composition as claimed in any one of the preceding claims wherein said hollow microsphere has a particle size from 100 to 600 nanometer.

12. A composition as claimed in any one of the preceding claims comprising 50 to 80% water.

13. A composition as claimed in any one of the preceding claims in the form of a lotion having a viscosity of 1500 to 6000 cP as measured by a Brookefield Viscosity meter using LV #4, 30 rpm, 30° C..

14. A method of providing enhanced SPF to skin comprising the step of applying a composition as claimed in any one of the preceding claims on to the desired surface of skin.

15. Use of a composition as claimed in any one of the preceding claims 1 to 13 for obtaining an SPF of at least 20.
